(19) United States
(12) Patent Application Publication
(10) Pub. No.: US 2008/0096188 A1
(43) Pub. Date: Apr. 24, 2008

(54) Method to predict degradation of a landfill

(75) Inventor: Wendy Jo Davis-Hoover, Wyoming, OH (US)

Correspondence Address: BROWDY AND NEIMARK, P.L.L.C.;624 NINTH STREET, NW, SUITE 300, WASHINGTON, DC, 20001-5303, US

(73) Assignee: U.S. Environmental Protection Agency EPA, Washington, DC (US)

(21) Appl. No.: 11/583,055

(22) Filed: Oct. 19, 2006

(51) Int. Cl. C12Q 1/70; C12Q 1/68

(52) U.S. Cl. 435/5; 435/6

(57) ABSTRACT

Determining when a landfill is stabilized by comparing the biological signature of a landfill over a predetermined time period with that of a known stabilized landfill.

### FIELD OF THE INVENTION

The present application relates to a method for determining the degradation, and therefore stability, of a landfill.

### BACKGROUND OF THE INVENTION

The safety of a particular landfill is determined by the degree of degradation it has yet to accomplish. The greater the extent of degradation, the more stable, and therefore less toxic or volatile the waste site. Heretofore, it has been difficult to determine the extent to which landfills have degraded, and, consequently, have attained an acceptable level of safety.

Previously, 16S rDNA sequences have been utilized to determine the presence of activated sludge bacterial strains in wastewater sludge, soil, or groundwater. (See U.S. Published Applications Nos. US 2003/0207321, 0207320, and 0203398, as well as U.S. Pat. No. 6,608,190, all to Bramucci et al.). Similarly, Ebsersole et al., in U.S. Pat. No. 6,894,156 and U.S. Published Application No. US 2005/0148015, provide a means for identifying dechlorinating bacteria based upon a unique 16S rRNA profile. Sandhu et al., in U.S. Pat. No. 6,180,339, disclose methods for identifying various disease-causing fungi in environmental samples, using nucleic acid probes and primers that detect rRNA, rDNA, or PCR products from a variety of fungi. U.S. Pat. Nos. 5,567,587, 5,601,984, 5,641,631 and 5,723,597 to Kohne provide means for detecting and quantitating organisms, both prokaryotes and eukaryotes, that contain rRNA or other RNA. The method can determine the state of growth of a microorganism, and can be used to detect and quantify previously unknown organisms. Crocetti et al., in U.S. Published Application No. US 2003/0170654, disclose probes and primers for the detection of polyphosphate accumulating organisms in wastewater. The process employs oligonucleotide probes or primers having a sequence of at least 12 nucleotides that is unique to the 16S rDNA of the polyphosphate accumulating organisms.

### SUMMARY OF THE INVENTION

It is an object of the present invention to provide an inexpensive and accurate means to determine and quantify live or dead microorganism, including but not limited to fungi, viruses, and bacteria in a landfill by utilizing various applicable molecular biology techniques.

It is a further object of the present invention to compare, over time, the biological signature of landfill bacteria to determine the extent to which a particular landfill has degraded, and therefore stabilized.

A still further object of the instant invention is to measure 16S rRNA within a landfill to determine a molecular signature for the microorganisms or flora therein.

It is a still further object of the present invention to quantify the microorganisms or flora within a landfill, based upon the molecular signature of said bacteria or flora's 16S rRNA.

It is a further object of the instant invention to measure 16S rRNA in a landfill leachate or in other waste products to quantify live bacteria or flora therein.

It is a further object of the instant invention to determine the stability, and therefore safety, of a particular landfill based upon the quantity of microorganisms or flora measured therein.

It is a still further object of the present invention to provide a means for determining the safety, stability, and extent of degradation of municipal solid waste landfills, as well as more specialized landfills, such as those from bioreactors, construction, demolition, or the like.

The scope and content of the present invention is not intended to be limited by or to the above mentioned objects.

The inventor present inventor has found that measuring the biological signature of various flora/microorganisms present in a landfill provides an inexpensive method for determining the degree of stabilization of said landfill. More particularly, the 16S Ribosomal RNA (16S rRNA) present in a landfill or leachate therefrom may be used to quantify the live or dead microorganisms or flora therein.

The process utilizes molecular methods to evaluate the characteristics of one or more selected molecular signatures within the landfill. Measured over time, comparison of these signatures provides an accurate and inexpensive means of determining when a landfill reaches its greatest stability, and therefore safety. The process applies to municipal waste, as well as landfills generated by bioreactors, demolition, construction, and other specialized landfills.

### DETAILED DESCRIPTION OF THE INVENTION

Degradation of a landfill can be projected by comparing the biological signature of a landfill over a predetermined time period to determine the characteristics of the signature during the stabilization process and when the landfill is stabilized. Once these characteristics of a stabilized landfill are determined, degradation of other landfills can be compared with these characteristics to predict when those other landfills will be stabilized.

Molecular biology methods are used to determine the normal flora of a stable landfill. More particularly, S ribosomal RNA in a landfill is measured to quantify microorganisms in a landfill.

Measuring S Ribosomal RNA in a landfill leachate or other waste products can be used to quantify microorganisms in a landfill.

Methods for identifying microorganisms involving the use of DNA probes based on the sequences of rRNA molecules can be used to test a sample for many different organisms rapidly and accurately (Husse et al., J. Biotechnol. 47(1):3-38, 1996). All cells contain ribosomes. Each ribosome is composed of three distinct rRNA molecules and a variety of protein molecules. In bacteria, the medium sized rRNA molecule, i.e., the 16S rRNA molecule, is particularly useful for identifying bacteria. The nucleotide sequence of the 16S rRNA molecule has conserved regions that are present in most if not all bacteria and variable regions that can be used to distinguish species and subspecies. Since an rRNA molecule is a direct gene product that results from transcription of a corresponding RNA gene (rDNA), rDNA can be specifically and rapidly isolated from a particular microorganism or a mixture of microorganisms by using appropriate DNA primers and the polymerase chain reaction (PCR) to amplify the rDNA. The pattern of fragments resulting form cutting the PCR product with a set of restriction endonucleases can be used to identify the organism from which the rDNA was amplified. Alternatively, in situ hybridization techniques are known whereby fluorescent probes based on specific 16S rRNA sequences can be used to demonstrate the presence of specific bacteria in samples of sludge (Wagner et al., Appl. Environm. Microbiol. 59:1520-1525, 1993).

In one embodiment, fluorescent labeled PCR product is run on a 1% agarose gel and the product quantified by image analysis as described in Kerkhof et al., Marine Biol. Biotech. 6(3): 260-267, 1997. PCR product is digested with MnII endonuclease for 16S rRNA amplicons. All digests are in 20 microliter volumes for six hours at about 37° C. DNA is precipitated by adding 2.3 microliters of 0.75 M sodium acetate and 5 micrograms glycogen with 37 microliters of 95% ethanol. Precipitated DNA is washed with 70% ethanol and dried briefly. The dried DNA is resuspended in 19.7 microliters of deionized from amide and 0.3 microliters of ROX 500 size standard for 15 minutes before analysis. Peak detection is set at 25 arbitrary fluorescent units. For comparative analysis, all peaks within a fingerprint are normalized to the total area for that sample. The presence of absence of a T-RFLP peak is used to compare any two samples with a Bray-Curtis similarity index:

SIM_{if}=2Σ min(x_{ik′} xⱼₖ)/Σ(xⱼₖ+xᵢₖ) for k=1-S

where S is the number of terminal restriction fragments (t-RFs) and xᵢₖ is the abundance (peak area) of the T-RF (k) in sample i. The comparative Bray-Curtis index is calculated for all sample pairs of the normalized profiles using the Combinatorial Polythetic Agglomerative Hierarchical clustering package.

Examples of Gram negative bacteria that can be detected and/or whose nucleic acid can be isolated using the kits and methods of the invention include but are not limited to Gram negative rods (e.g., anaerobes such as bacteroidaceae (e.g., Bacteroides fragilis), facultative anaerobes, enterobacteriaceae (e.g., Escherichia coli), vibrionaceae (e.g., Vibrio cholerae), pasteurellae (e.g., Haemophilus influenzae), and aerobes such as pseudomonadaceae (e.g., Pseudomonas aeruginosa); Gram negative cocci (e.g., aerobes such as Neisseriaceae (e.g., Neisseria meningitidis) and Gram negative obligate intracellular parasites (e.g., Rickettsiae), (e.g., Rickettsia spp.). Examples of Gram negative bacteria families that can be detected and/or whose nucleic acid can be isolated include but are not limited to Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae and Spirochaetaceae.

Examples of Gram positive bacteria that can be detected and/or whose nucleic acid can be isolated using the kits and methods of the invention include but are not limited to A. globiformis, B. subtilis, C. renale, M. luteus, R. erythropolis, Ea39, Ben-28 and S. lividans. Gram positive bacteria that can be detected and/or whose nucleic acid can be isolated also are in groups that include, for example, Corynebacterium, Mycobacterium, Nocardia; Peptococcus (e.g., P. niger); Peptostreptococcus (e.g., Ps. anaerobius; some species in the group form clumps and clusters); some species in the group form diplococci (the latter of which are distinguished by their ability to form butyrate); and some species in the group are capable of fermentation, reduction of nitrate, production of indole, urease, coagulase or catalase); Ruminococcus; Sarcina; Coprococcus; Arthrobacter (e.g., A. globiformis, A. citreus or A. nicotianae); Micrococcus (e.g., M. luteus (previously known as M. lysodeikticus), M. lylae, M. roseus, M. agilis, M. kristinae and M. halobius; Bacillus (e.g., B. anthracis, B. azotoformans, B. cereus, B. coagulans, B. israelensis, B. larvae, B. mycoides, B. polymyxa, B. pumilis, B. stearothormophillus, B. subtilis, B. thuringiensis, B. validus, B. weihenstephanensis and B. pseudomycoides); Sporolactobacillus; Sporocarcina; Filibacter; Caryophanum and Desulfotomaculum. Other Gram positive bacteria that can be detected and/or whose nucleic acid can be isolated fall into the group Clostridium, which often include peritrichous flagellation, often degrade organic materials to acids, alcohols, CO₂, H₂ and minerals (acids, particularly butyric acid, are frequent products of clostridial fermentation), and in one aspect form ellipsoidal or spherical endospores, which may or may not swell the sporangium. Species of Clostridium that can be detected and/or whose nucleic acid can be isolated include psychrophilic, mesophilic or thermophilic species, saccharolytic species, proteolytic species and/or specialist species, and those that are both saccharolytic and proteolytic species. Saccharolytic species of Clostridium that can be detected and/or whose nucleic acid can be isolated include but are not limited to Cl. aerotolerans, Cl. aurantibutyricum, Cl. beijerinckii, Cl. botulinum B, E, F*, Cl. butyricum, Cl. chauvoei, Cl. difficile, Cl. intestinale, Cl. novyi A, Cl. pateurianum, Cl. saccharolyticum, Cl. septicum, Cl. thermoaceticum, and Cl. thermosaccharolyticum.

Proteolytic species of Clostridium that can be detected and/or whose nucleic acid can be isolated include but are not limited to Cl. argeninense, Cl. ghoni, Cl. limosum, Cl. putrefaciens, Cl. subterminale and Cl. tetani. Species that are proteolytic and saccharolytic that can be detected and/or whose nucleic acid can be isolated include but are not limited to Cl. acetobutylicum, Cl. bifermenans, Cl. botulinum A, B, F (prot.)*, Cl. botulinum C, D*, Cl. cadaveris, Cl. haemolyticum, Cl. novyi B, C, *Cl. perfringens, Cl. putrefaciens, Cl. sordelli and Cl. sporogenes. As indicated by an asterisk, Cl. botulinum is subdivided into a number of types according to the serological specificities of the toxins produced. Specialist Clostridium species that can be detected and/or whose nucleic acid can be isolated include but are not limited to Cl. acidiurici, Cl. irregularis, Cl. kluyveri, Cl. oxalicum, Cl. propionicum, Cl. sticklandii and Cl. villosum. These specificities are based on neutralization studies. Other Clostridium species that can be detected and/or whose nucleic acid can be isolated include those that produce botulinum toxins.

Examples of fungi that can be detected and/or whose nucleic acid can be isolated using the kits and methods of the invention include but are not limited to Halocyphina villosa, Hypoxylon oceanicum, Verruculina enalia, Nia vibrissa, Antennospora quadricornuta, Lulworthia spp. and Aigialus parvus. Examples of algae that can be detected and/or whose nucleic acid can be isolated include but are not limited to brown algae (e.g., Phylum Phaeophycota Dictyota sp.); (Class Phaeophyceae, Family Dictyotaceae); green algae (e.g., Phylum Chlorophycota Chaetomorpha gracilis (Class Chlorophyceae, Family Cladophoraceae); and red algae (e.g., Phylum Rhodophycota, Catenella sp. (Class Rhodophyceae, Family Rhabdoniaceae).

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

What is claimed is:

1. A method for determining when a landfill is stabilized comprising measuring the biological signature of the landfill over a predetermined time period to determine the characteristics of the signature, and comparing the signatures over the predetermined period of time to the signatures of a known stabilized landfill.

2. The method according to claim 1 wherein the biological signature is measured using molecular biology methods.

3. The method according to claim 2 wherein microorganisms are measured in the landfill by measuring Ribosomal RNA.

4. The method according to claim 1 wherein the biological signatures include at least one of anaerobic, aerobic, sulfate reducing, denitrifying, methanogenic, and methanotrophic bacteria, fungi, and viruses.

5. A method for quantifying microorganisms in a landfill comprising measuring Ribosomal RNA in a landfill leachate.
